(19) United States
(12) Patent Application Publication
(10) Pub. No.: US 2017/0079704 A1
(43) Pub. Date: Mar. 23, 2017

(54) SURGICAL INSTRUMENT, SURGICAL KIT AND METHOD FOR MANUFACTURING THE SAME

(71) Applicant: IN2BONES, Ecully (FR)

(72) Inventors: Christian Baertich, Toulouse (FR); Joël Vernois, Picquigny (FR); Adrien Ray, Carouge (CH); David Jonathan Redfern, Hove (GB)

(73) Assignee: IN2BONES, Ecully (FR)

(21) Appl. No.: 15/267,781

(22) Filed: Sep. 16, 2016

(30) Foreign Application Priority Data
Sep. 17, 2015 (FR) FR-15 58776

(51) Int. Cl. A61B 17/88; A61B 17/68

(52) U.S. Cl. CPC A61B 17/8866; A61B 17/68; A61B 2017/681

(57) ABSTRACT

The invention concerns a surgical instrument (1) intended to allow modifying the relative positioning of bone bodies of a patient, for example of his foot, said surgical instrument (1) including:
a gripping handle (5) extending along a first longitudinal axis (A-A′),
a working head, including an elongate body extending along a second longitudinal axis between a first end linked to the gripping handle (5) and a second free end (11), said body presenting an upper surface (12) and a lower surface linking said first and second (11) ends,
said surgical instrument (1) being characterized in that the working head is provided with an axial groove (16) formed in said upper surface (12).

Surgical instruments intended to perform a modification of the relative positioning of bone bodies.

The present invention concerns the field of surgical instrumentation, and in particular surgical tools intended to perform a modification of the relative positioning of bone bodies of a patient, in particular in the context of the treatment of the foot, and to be inserted, to this end, between the considered bone bodies.

More particularly, the present invention concerns a surgical instrument intended to be maneuvered like a lever in order to allow modifying the relative positioning of bone bodies of a patient, for example of his foot, said surgical instrument including:
a gripping handle extending along a first longitudinal axis A-A′,
a working head, including an elongate body extending along a second longitudinal axis B-B′ between a first end linked to the gripping handle and a second free end, said body presenting an upper surface and a lower surface linking said first and second ends.

The present invention also concerns a method for manufacturing such a surgical instrument.

The present invention further concerns a surgical kit intended for the implementation of an operative technique, preferably percutaneous, in particular in the context of the treatment of a pathology of the foot of a patient, comprising such a surgical instrument.

In the context of the treatment of some bone pathologies, in particular of the foot of a patient, it is sometimes necessary to the surgeon to spread apart and reposition bone bodies relative to each other with the aim of allowing their osteosynthesis in a given preferred anatomical configuration.

In particular, in the case of the treatment of a pathology such as the hallux valgus, which is a relatively common pathology characterized by a deformation of the foot corresponding to the deflection of the first metatarsus in varus and of the big toe (hallux) in valgus, a well-known operative technique consists in practicing an osteotomy of the head (distal metaphysis) of the first metatarsus, that is to say to surgically dissect the first metatarsus, in order to reorientate, manually or using a specific surgical instrument, the head and its articular surface and therefore correct the deformation. A wire is sometimes introduced axially in the body (diaphysis) of the metatarsus in order to hold the bone segments in the corrected position during their osteosynthesis.

Conventionally, this surgical operative technique is carried out in [symbol] open [symbol] surgery, that is to say by practicing large incisions in the skin and the soft tissues so as to allow the surgeon to access to the area to be treated, easily and with a good visual control. Being relatively heavy and imposing a hospitalization of the patient, this operative technique is progressively replaced by a less invasive method which allows an ambulatory operation: the percutaneous or [symbol] mini-invasive [symbol] surgery.

More complex to handle by the surgeon and requiring special surgical tools which are often expensive, this technique is practiced through the skin generally under regional anesthesia and radiographic control, throughout a few incisions with a length of about 2 to 5 mm only, called the first pathways, intended to allow the introduction in the body of the patient of instruments and possible surgical implants necessary to the surgical operation.

In the field of surgical instruments intended to perform such a correction of the relative positioning of bone bodies of a patient, and in particular of segments of a bone of the foot, there are known lever-type manual instruments, also called retractors, consisting of a rod terminated by a thinner and curved head so as to form a [symbol] Z [symbol] shape with the rod. These retractors are made exclusively of a metallic material, in particular by machining and bending.

Once the first metatarsus is dissected, the bone segments which constitute on the one hand the head and on the other hand the body of the first metatarsus are spread apart by inserting the tip of the retractor in the medullary cavity of the body of the first metatarsus and by using the retractor as a lever bearing on the head of the first metatarsus and on the head of the bone of the big toe. In order to improve the support, some of the known instruments further present a head provided with an enlarged and substantially flat area.

While such instruments generally give satisfaction, they however remain not very ergonomic and in particular they are not adapted to surgical operations requiring the implementation of some specific instruments, in particular of an osteosynthesis wire to ensure the fusion of the bone segments.

Indeed, setting such a wire imposes to the surgeon to remove his instrument from the medullary cavity of the first bone segment beforehand and therefore release the spreading effort exerted on the bone bodies. As a result, the positioning of an osteosynthesis wire turns out to be relatively complex and inaccurate.

Consequently, the invention aims to remedy to the different drawbacks enumerated before, and to propose a surgical instrument which is more polyvalent and in particular intended to allow modifying the relative positioning of bone bodies of a patient, allowing the simultaneous implementation of an osteosynthesis wire in a particularly accurate manner.

Another object of the invention aims to propose a new surgical instrument, intended to allow modifying the relative positioning of bone bodies of a patient, which allows the accurate implementation of an osteosynthesis wire, regardless of its diameter.

Another object of the invention aims to propose a new surgical instrument, intended to allow modifying the relative positioning of bone bodies of a patient, which is particularly ergonomic and easy to manipulate.

Another object of the invention aims to propose a new surgical instrument, intended to allow modifying the relative positioning of bone bodies of a patient, which is safe and relatively inexpensive to manufacture.

Another object of the invention aims to propose a new method for manufacturing a surgical instrument, which is both simple and inexpensive to implement.

Another object of the invention aims to propose a new surgical kit intended for the implementation of an operative technique, preferably percutaneous, which is complete, ergonomic, safe and inexpensive.

The objects assigned to the invention are achieved by means of a surgical instrument intended to be maneuvered like a lever in order to allow modifying the relative positioning of bone bodies of a patient, for example of his foot, said surgical instrument including:
a gripping handle extending along a first longitudinal axis A-A′,
a working head, including an elongate body extending along a second longitudinal axis B-B′ between a first end linked to the gripping handle and a second free end, said body presenting an upper surface and a lower surface linking said first and second ends,

said surgical instrument being characterized in that the working head is provided with an axial groove formed in said upper surface.

The objects assigned to the invention are also achieved by means of a method for manufacturing such a surgical instrument, characterized in that said method comprises one single molding step during which said surgical instrument is integrally made in one single piece.

The objects assigned to the invention are also achieved by means of a surgical kit intended for the implementation of an operative technique, preferably percutaneous, in particular in the context of the treatment of a pathology of the foot of a patient, comprising such a surgical instrument, and being characterized in that it also comprises at least one bistoury blade holder handle, a rugine and a rasp.

Other objects and advantages of the invention will appear in more details upon reading the description that follows, as well as with reference to the appended drawings, provided only for an explanatory and non-restrictive purpose, in which:

FIG. 1 illustrates, in a profile view, a surgical instrument in accordance with the invention;

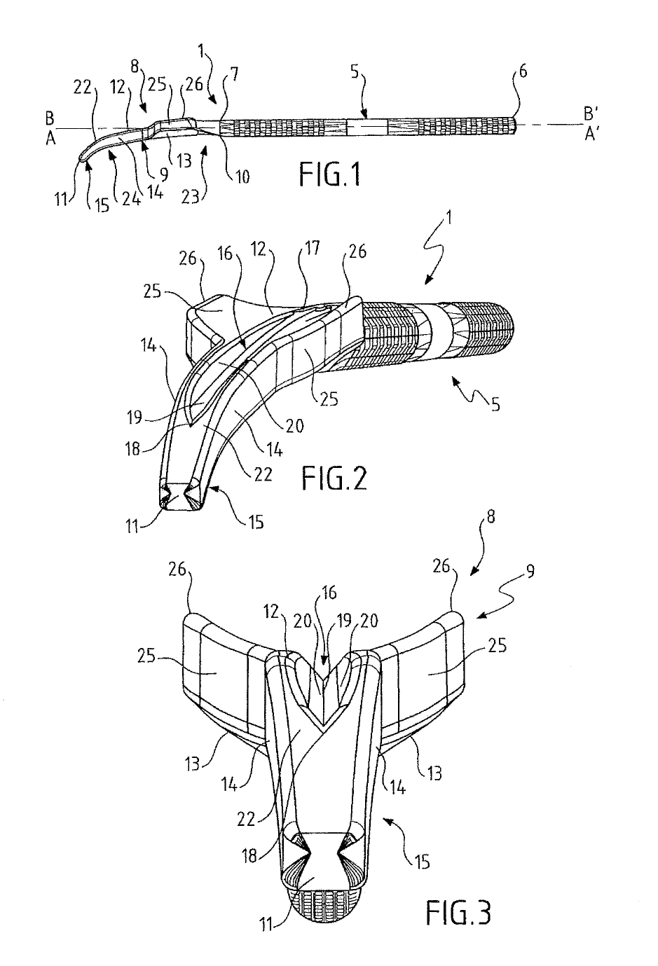

FIGS. 2 and 3 illustrate, respectively in a perspective and in a front views, the instrument of FIG. 1, so as to particularly highlight the respective configurations of the groove and the fins provided on the working head of this instrument;

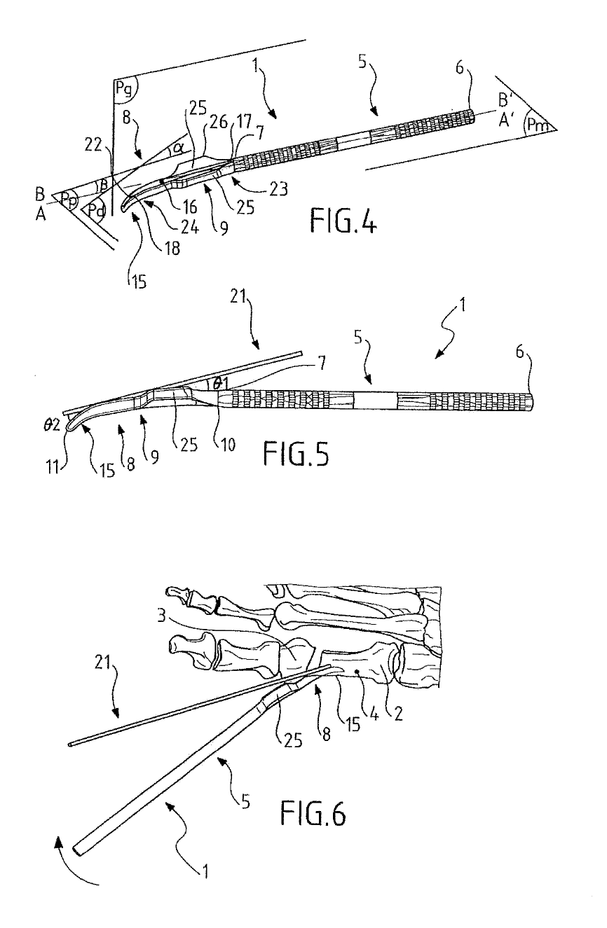

FIG. 4 illustrates, in a perspective view, the surgical instrument of FIGS. 1 to 3, so as to particularly highlight its curvature features, and in particular, the relative inclinations of the gripping handle, the working head and its working tip;

FIG. 5 illustrates, in a profile view, an example of a conjugate implementation of the surgical instrument of the invention and an osteosynthesis wire;

FIG. 6 illustrates an example of a conjugate implementation of the surgical instrument of the invention and an osteosynthesis wire in connection with bone bodies in the case of a treatment of a pathology of the foot of a patient by osteotomy;

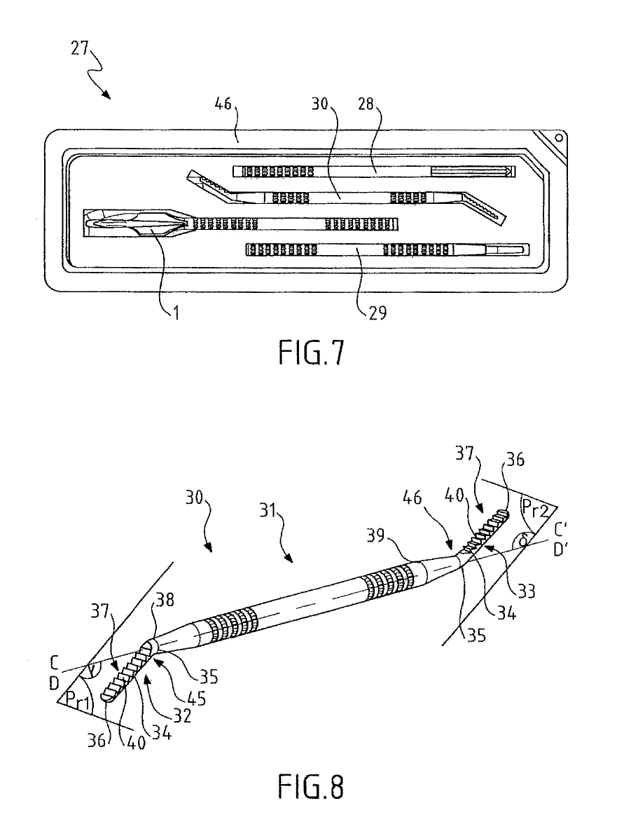

FIG. 7 illustrates, in a top view, a preferred embodiment of a surgical kit in accordance with the invention;

FIG. 8 illustrates, in a profile view, an embodiment of a rasp as preferably included in the surgical kit of FIG. 7;

FIGS. 9 to 12 illustrate different possible geometric configurations of the teeth of the rasp of FIG. 8.

The invention concerns a surgical instrument 1, advantageously of the retractor type, intended to be maneuvered like a lever in order to allow modifying the relative positioning of bone bodies 2, 3 of a patient, for example of his foot, one embodiment of which in accordance with the invention is illustrated in FIGS. 1 to 5. In this instance, the surgical instrument 1 of the invention is particularly adapted to be used by a surgeon during a percutaneous surgical operation on a patient, in particular in the context of the treatment of a pathology of the foot by osteotomy, for example a hallux valgus.

By [symbol] bone bodies [symbol] , are meant bones, cartilages, fragments of bones or cartilages, preferably intended to be fusioned by osteosynthesis after having undergone a modification of their relative positioning using the surgical instrument 1. Still more preferably, the invention takes on its full sense when these bone bodies 2, 3 are two in number and are constituted by separate segments of the same bone, typically the body of a bone fitted with a medullary cavity 4 and its head. Moreover, by the modification of the positioning of bone bodies 2, 3, is meant the fact of displacing said bone bodies 2, 3, preferably using the instrument 1 of the invention, in order to change their relative orientation or positioning, and this for example, with the aim of rectifying a bone alignment for therapeutical or plastic purposes.

Of course, without departing from the scope of the invention, the surgical instrument 1 may be used in the context of the treatment of a pathology affecting bones others than those of the foot, for example bones of the hand, as long as it is necessary to displace bone bodies 2, 3 in order to modify or correct their positioning. Without restriction to an exclusive use on a human patient, the surgical instrument 1 may also be implemented in the context of an animal surgery for the treatment of similar bone pathologies.

According to the invention, and as illustrated in FIG. 1, said surgical instrument 1 includes a gripping handle 5, preferably for manual gripping, extending along a first longitudinal axis A-A′. Indeed, the surgical instrument 1 is preferably intended for manual gripping, that is to say that it can be maneuvered directly by hand by the surgeon. Nonetheless, nothing precludes the possibility of implementing the instrument 1 in a manner other than manually, for example using a robotic means. Preferably having a cylindrical shape, this handle 5 advantageously extends longitudinally between a proximal end 6 and a distal end 7.

The surgical instrument 1 of the invention also includes a working head 8, the latter including an elongate body 9 extending along a second longitudinal axis B-B′, between a first end 10 linked to the gripping handle 5, preferably at the level of the distal end 7 of the latter, and a second free end 11. Advantageously, said working head 8 extends in the extension of the gripping handle 5, said first A-A′ and second B-B′ axes being preferably coincident. Nonetheless, it is possible to consider an alternative configuration, not illustrated in the figures, in which said working head 8 would not extend in the extension of the gripping handle 5, but would on the contrary be linked to said handle 5 in an offset manner. Furthermore, said first A-A′ and second B-B′ axes are advantageously comprised in the same plane.

Preferably, said working head 8 is firmly linked at its first end 10 to the gripping handle 5, said working head 8 and said gripping handle 5 being made secured to each other. The working head 8 may constitute a part independent from the gripping handle 5, possibly interchangeable, attached and firmly held to the latter for example by bonding, screwing, clipping or still by plugging. Nonetheless, preferably, said gripping handle 5 and said working head 8 are integral with each other, that is to say that they advantageously constitute one single piece completely made of a given material.

Said body 9 presents an upper surface 12 and a lower surface 13 linking said first 10 and second 11 ends. These upper 12 and lower 13 surfaces form the outer contour of the working head 8 and its body 9, exclusively between said first 10 and second 11 ends. These upper 12 and lower 13 surfaces do not include the first 10 and second 11 ends.

Preferably, the body 9 of the working head 8 extends:
laterally between two lateral edges 14, and
transversely between said upper 12 and lower 13 surfaces,

said lateral edges 14 and said upper 12 and lower 13 surfaces being respectively substantially parallel over at least a portion of the length of the working head 8, and then convergent towards the free end 11 of the latter so as to form at this free end 11 a working tip 15. In other terms, if the body 9 of the working head 8 presents a rectangular section, the working tip 15 presents, in turn, a rectangular section, but with a surface area smaller than the surface area of the section of the working head 8. In this manner, and as illustrated in the figures, the working head 8 presents, at least at the level of its working tip 15, a particular slender shape capable, for example, of being inserted in a narrow incision realized in the skin and in the soft tissues in the context of a percutaneous-type surgical operation, so as to bring the working tip 15 to cooperate with the medullary cavity 4 of a bone body 2, for example of a bone segment of the first metatarsus.

According to a major feature of the invention, the working head 8 is provided with an axial groove 16, advantageously rectilinear, that is to say extending along the second longitudinal axis B-B′ of the body 9 of the working head 8, which is formed in said upper surface 12. Advantageously, said axial groove 16 extends between a proximal opening 17 and a distal opening 18, said proximal 17 and distal 18 openings opening from the upper surface 12 of the body 9 of said working head 8. Hence, said groove 16 does not constitute a closed slot, but rather a kind of a gutter open at each of its ends.

Preferably, said groove 16 is formed by:
a groove bottom 19 extending, from said proximal opening 17, at the level of which the working head 8 is linked to the gripping handle 5, along the longitudinal axis B-B′ over at least a portion of the length of the working head 8,
two groove walls 20 rising from the bottom 19 up to the upper surface 12.

As illustrated in the figures, the proximal opening 17 of the groove 16 is preferably coincident with the first end 10 of the body 9 of the working head 8, so that said groove 16 extends from the first end 10 over a more or less long portion of the length of the working head 8. Thus, the groove 16 is advantageously capable of receiving and guiding an elongate and thin body, such as an osteosynthesis wire 21, for example Kirschner wires, this may be fit and slide in said groove 16 at either side of these proximal 17 and distal 18 openings, as illustrated in FIG. 5. The wire 21 then forms, respectively at the level of said proximal 17 and distal 18 openings, an angle Θ1, Θ2 with the working head. Moreover, in practice, the length of the groove 16 along the longitudinal axis B-B′ allows the surgical instrument 1 to receive and accurately guide rigid and long wires 21. However, in the preferred case illustrated in particular in FIGS. 2 and 3, said groove 16 extends towards the free end 11 of the working head 8 only up to the vicinity of the base 22 of the working tip 15, so as not to mechanically weaken said working tip 15.

Still more advantageously, and as illustrated in particular in FIGS. 2 and 3, said walls 20 are configured so as to confer to said groove 16 a substantially conical profile, that is to say that the walls 20 are preferably inclined with respect to each other towards the groove bottom 19 so as to form a [symbol] V [symbol] -shaped profile. Such a configuration is particularly interesting, because it allows the groove 16 to receive and guide almost as accurately wires 21 with different diameters, typically diameters comprised between 1.0 and 2.5 mm, said wires 21 coming into contact with the walls 20 more or less deeply in the groove 16.

Moreover, in the preferred embodiment illustrated in the figures, said groove 16 presents a tapered shape, its walls 20 converging towards the proximal opening 17.

The surgical instrument 1 being intended to be maneuvered by the surgeon like a lever in order to allow the relative displacement of bone bodies 2, 3, the working head 8 is not inscribed in the average extension plane Pm of the gripping handle 5, in which the first longitudinal extension axis A-A′ of said handle 5 is inscribed, but it presents on the contrary a curved shape. Nonetheless, and in contrast with conventional retractor-type instruments, the working head 8 of the surgical instrument 1 is not configured so as to form a recurved [symbol] Z [symbol] shape with the handle 5. Indeed, as illustrated in FIGS. 1 to 4, said working head 8 is preferably recurved, that is to say that it presents a continuous curved general shape, without any change in direction, both the body 9 and the working tip 15 of the working head 8 tending to spread away from the average extension plane Pm of the gripping handle 5. Thus, and as illustrated in FIG. 4, the body 9 of the working head 8 preferably extends substantially along a proximal extension plane Pp secant to the first longitudinal axis A-A′, except its working tip 15 which in turn advantageously extends substantially according to a distal extension plane Pd secant to the proximal extension plane Pp. Furthermore, said working tip 15 preferably extends along an average longitudinal axis comprised in the same plane as the second axis B-B′. Thus, the body 9 of the working head 8 and the gripping handle 5 are linked by a first curvature area 23, whereas said working tip 15 and said working head 8 are advantageously linked by a second curvature area 24. Thus, the working head 8 presents a dual-inflection, the working tip 15 being bent with respect to the rest of the body 9 of the working head 8 and said body 9 being in turn bent with respect to the gripping handle 5, the first 23 and second 24 inflection areas being advantageously located respectively at the level of the first end 10 of the working head 8 linked to the gripping handle 5 and at the level of the base 22 of the working tip 15. More specifically, the body 9 of the working head 8 is inclined with respect to the gripping handle 5 so that the proximal extension plane Pp and the first longitudinal axis A-A′ are secant to each other so as to form a first angle of elevation a advantageously comprised between 30° and 60°, and more preferably equal to about 45°, whereas the working tip 15 is inclined with respect to the body 9 of the working head 8 so that the distal extension plane Pd and the proximal extension plane Pp are secant to each other so as to form a second angle of elevation β advantageously comprised between 30° and 60°, and more preferably equal to about 45°.

In a particularly advantageous manner, said proximal opening 17 and distal opening 18 of the groove 16 opening from the upper surface 12 of the body 9 of said working head 8 are positioned respectively at the level of said first 23 and second 24 curvature areas. In this manner, as illustrated in FIG. 6, the combination of such a recurved configuration of the working head 8 of the instrument 1 with said axial groove 16, advantageously with a conical profile, thereby allows displacing two bone segments or bodies 2, 3 relative to each other by a simple lever movement and fastening the osteosynthesis thanks to a wire 21 without having to remove the instrument 1 since the latter can slide freely in said groove 16 formed in the upper surface 12 of the working head 8, the lever function allowed by the curvature of the working head 8 not hindering the guide function offered by the groove 16. Consequently, besides ensuring its primary function of displacing bone segments or bodies 2, 3, the instrument 1 allows a simultaneous, easy and more accurate positioning of said wire 21, while complying with the orientation chosen by the surgeon.

Preferably, and as illustrated in FIGS. 1 to 5, the working head 8 of the surgical instrument 1 is fitted with two longitudinal flanges 25. These flanges 25 protrude from the elongate body 9 of said working head 8 so as to prolong said upper 12 and lower 13 surfaces, these being disposed symmetrically with respect to an extension plane Pg of the groove, preferably orthogonal to the distal extension plane Pd of the working head 8, and radially with respect to the longitudinal axis B-B′. In a particularly advantageous manner, and as illustrated in particular in FIGS. 2 and 3, said longitudinal flanges 25 are incurved so as to locally confer to said upper surface 12 a substantially concave shape and to said lower surface 13 a substantially convex shape. In other terms, the flanges 25 are configured relatively to the body 9 of the working head 8, so that their longitudinal edges 26, preferably parallel, rise away from the distal extension plane Pd of the working head 8 and, advantageously, in a direction opposite to that of the general curvature of said working head 8 described hereinbefore. In this manner, and taking once again the example, illustrated in FIG. 6, of a treatment of a pathology of the foot of a patient by osteotomy, when the working tip 15 of the instrument 1 is inserted in the medullary cavity 4 of a first bone segment or body 2, for example of a first metatarsus, the upper surface 12 provided with flanges 25 advantageously conform to the contour of a second bone segment or body 3, for example the head of this first metatarsus, so as to offer a wide and stable support to the instrument 1 when the latter is maneuvered like a lever in order to modify the positioning of these first 2 and second 3 bone bodies. Thus, the instrument 1 is particularly ergonomic, practical and safe to use, since the risk of slipping, or skidding, of the instrument 1 on the bone bodies 2, 3, and in particular on the second bone segment or body 3, under the lever effort exerted by the surgeon is particularly limited.

While not necessarily being intended to be inserted in the body of the patient, in the case of a percutaneous implementation, said longitudinal flanges 25 are preferably positioned proximate to said first end 10 of the working head 8, that is to say the end at the level of which said head 8 is linked to the handle 5, and extending longitudinally substantially over half the length of said working head 8. This advantageously allows preserving a narrow and sufficiently long portion of the length of the working head 8 devoid of the flanges 25 so as to be inserted in the body of the patient through an incision, with a length of about 2 to 5 mm, and therefore allow the instrument 1 to effectively act as a lever,

Preferably, the above-described surgical instrument 1 is intended for a single use, that is to say that it is intended to be destroyed or recycled/valorized at the end of its use for a given patient and surgical operation. This allows not only a greater sanitary safety, but also a significant economic gain for the surgeon and the healthcare center in which he exerts, the costs related to the cleaning, sterilization and reconditioning of reusable surgical instruments being particularly high compared to the cost of the instrument itself. As such, said surgical instrument 1 is advantageously made of a polymer material, for example a polyacrylamide (PAA) based composite polymer material, the latter being optionally loaded, such as for example a polyacrylamide-based reinforced polymer material of the range [symbol] IXEF [symbol] ®commercialized by the company SOLVAY. Indeed, such a material has the advantage of being a biocompatible material having a good resistance to fatigue and a good tenacity. Optionally, it may be loaded with fibers, for example carbon or glass fibers, so as to confer to the surgical instrument 1 a great rigidity and an excellent mechanical bending strength. In addition, polyacrylamide-based materials are known to be particularly easy to implement, in particular by injection molding, even with a high fiber content. They present a small shrinkage at molding and therefore allow the accurate and repeatable realization of parts with small dimensions and thicknesses and complex shapes.

The invention further concerns, as such, a method for manufacturing a surgical instrument 1 as described before in its variant according to which its gripping handle 5 and its working head 8 are integral with each other, said method comprising one single molding step, for example an injection molding step, during which said surgical instrument 1 is integrally made in one single piece. In other terms, the manufacturing method of the invention allows making said surgical instrument 1 integrally in one piece, in one single operation, without any other finishing operation such as machining or others. Advantageously, the material intended to be molded during the manufacturing method is a polymer material, preferably a polyacrylamide (PAA) based composite polymer material as mentioned hereinbefore.

The invention also concerns, as such, a surgical kit 27 intended for the implementation of an operative technique, preferably percutaneous, in particular in the context of the treatment of a pathology of the foot of a patient, comprising a surgical instrument 1 in accordance with the invention, and as described hereinbefore, and also comprising at least one bistoury blade holder handle 28, a rugine 29 and a rasp 30.

The blade-holder handle 28 is capable of receiving a bistoury blade (not represented) in a removable manner. In the example case of a surgical kit 27 intended for the realization of percutaneous surgical operations, this blade-holder handle 28 equipped with a bistoury blade is intended to allow the realization of very thin incisions of the skin and soft tissues constituting first pathways through which will be introduced in the body of the patient the instruments and the possible surgical implants necessary to the surgical operation.

The rugine 29 is a surgical tool intended to scrape off a bone surface and, in particular, to separate from the bones the surrounding soft tissues such as muscles or tendons, in order to enable working in direct contact with said bone surface. Such a rugine 29 is formed by a small plate supported by a handle. Said plate is provided with sharp ridges and its free end, which forms a ridge perpendicular to the main axis of the handle, is beveled so as to form a leading edge specifically aggressive in order to ensure the detachment of the periosteum and the soft tissues.

Preferably, said plate and said handle form one single piece. Nonetheless, said rugine 29 may possibly be formed by distinct plate and handle, said plate being inserted in the handle in a permanent or still in a removable manner.

The rasp 30 (or [symbol] excavator [symbol] ), one embodiment of which is illustrated in FIG. 8, is a surgical tool which allows extracting from the body of the patient biological debris or sequestrum, constituted in particular by bone or tissular fragments mixed with blood, in particular those generated during an osteotomy operation, for example of the first metatarsus, or during a reduction of the volume of the metatarsal head.

Preferably, such a rasp 30 includes:
on the one hand, a rasp handle 31 extending along a third longitudinal axis C-C′, and
on the other hand, at least one rasp head 32, 33, including an elongate body 34 extending along a fourth longitudinal axis D-D′ between a first end 35 linked to the rasp handle 31 and a second free end 36, said body 34 presenting an active face 37 linking said first 35 and second 36 ends.

Advantageously, said at least one rasp head 32, 33 extends in the extension of the rasp handle 31, preferably from one of the ends 38, 39 of the latter, said third C-C′ and fourth D-D′ axes being preferably coincident.

Said active face 37 of the rasp head 32, 33 is provided with a row of parallel teeth 40, aligned along the fourth longitudinal axis D-D′, and presenting sharp ridges 41 capable of effectively rasping the bone bodies 2, 3 and the surrounding soft tissues in order to extract the sequestrum.

Conventionally, these teeth 40 are arranged in a [symbol] straight [symbol] configuration, illustrated in FIG. 9, that is to say that they extend linearly in a direction orthogonal to the fourth longitudinal axis D-D′, their front 42 and rear 43 faces being respectively inscribed in one single plane Pay, Par.

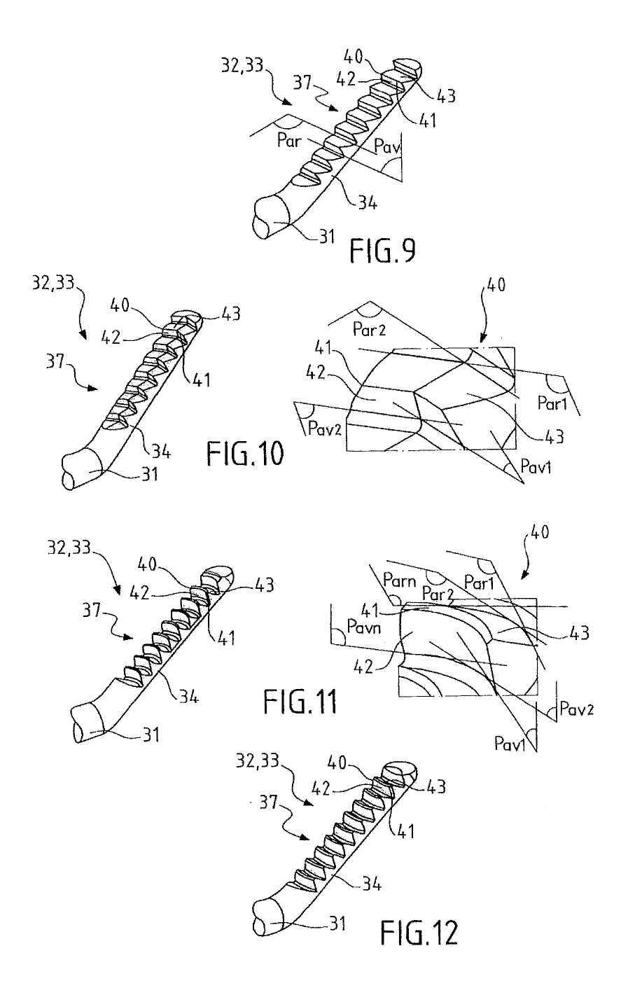

Preferably, the front 42 and rear 43 faces of said teeth 40 are respectively inscribed in at least two distinct secant planes Pay1, Pav2, Par1, Par2, so that said teeth 40 may advantageously present an alternative geometric configuration at least among the following ones:
a [symbol] chevron [symbol] type configuration: according to this first variant, illustrated in FIG. 10, the front 42 and rear 43 faces of said teeth 40 are inscribed respectively in two distinct secant planes Pav1, Pav2, Part Par2, so that said teeth 40 present a [symbol] VD [symbol] -shaped concave profile, the opening of the [symbol] V [symbol] shape preferably opening in the direction of the rasp handle 31;

a first [symbol] concave [symbol] configuration: according to this second variant, illustrated in FIG. 11, the front 42 and rear 43 faces of said teeth 40 are preferably inscribed respectively in a multitude of distinct secant planes Pav1, Pav2, . . . , Pavn, Par1, Par2, . . . , Parn, so that said teeth 40 present a curvilinear concave profile, the concavity of the teeth 40 opening preferably in the direction of the rasp handle 31;
a second [symbol] concave [symbol] configuration: according to this third variant, illustrated in FIG. 12, the front 42 and rear 43 faces of said teeth 40 are preferably inscribed in a multitude of distinct secant planes Pav1, Pav2, ,Pavn, Part Par2, . . . , Parn, so that said teeth 40 present a curvilinear concave profile, the concavity of the teeth 40 opening preferably in the direction opposite to that of the rasp handle 31.

Indeed, it has been observed that these [symbol] chevron [symbol] type and concave configurations, in comparison with the conventional [symbol] straight [symbol] configuration, advantageously allow blocking more bone sequestra in the concavity of each tooth and thus extracting them from the open wound more effectively and rapidly, which contributes to the reduction of the operative time.

Of course, the teeth 40 of said rasp head 32, 33, may present advantageous variants of the geometric configuration others than those described hereinabove, for example a configuration according to a [symbol] W [symbol] -shaped profile.

Preferably, the body 34 of said rasp head 32, 33 is firmly linked at its first end 35 to the rasp handle 31, said rasp head 32, 33 and said rasp handle 31 being made secured to each other. Thus, the rasp head 32, 33 may constitute a part independent from the rasp handle 31, possibly interchangeable, attached and firmly held to the latter for example by bonding, screwing, clipping or still by plugging.

Nonetheless, preferably, said rasp handle 31 and said rasp head 32, 33 are integral with each other, that is to say that they advantageously constitute one single piece completely made of a given material, preferably a polymer material, for. example a polyacrylamide (PAA) based composite polymer material, the latter being optionally loaded.

Preferably, and as illustrated in FIG. 8, said rasp 30 advantageously includes two rasp heads 32, 33, each linked to one of the ends 38, 39 of the rasp 30 handle.

The bodies 34 of the rasp heads 32, 33 extend respectively substantially along a first Pr1 and a second Pr2 extension plane secant to the third longitudinal axis C-C′, the bodies 34 of said rasp heads 32, 33 and the rasp handle 31 being respectively linked by a first 45 and a second 46 inflection area.

In a preferred variant illustrated in FIG. 8, said rasp 30 includes two rasp heads 32, 33. These extend respectively in an extension plane Pr1, Pr2, said planes Pr1, Pr2 being parallel and secant to the third longitudinal axis C-C′ so as to form with the latter a third angle of elevation γ, δ preferably comprised between 135° and 165°.

Furthermore, in this preferred variant, said rasp heads 32, 33 advantageously extend in opposite directions, thereby conferring to said rasp a [symbol] Z [symbol] -shape, so that the surgeon is not bothered by the presence of the other rasp head 32, 33, when he manually uses either one of the rasp heads 32, 33.

Nonetheless, other variants may be perfectly considered, the extension planes Pr1, Pr2 may be not parallel and the angles of elevation γ, δ respectively formed by these planes with the third longitudinal axis C-C′ may be identical or different, while advantageously being comprised between 135° and 165°.

Lastly, when said rasp 30 is in a variant in which it includes two rasp heads 32, 33, the teeth 40 of these may present geometric configurations, as described before, identical or still different, so as to offer to the surgeon a rasp 30 which is perfectly adapted to his needs.

It is understood that the above-described rasp 30, considered independently from said surgical kit 27, may constitute, as such, an invention on its own, distinct from the present invention.

Without departing from the scope of the invention, the surgical kit 27 may also include surgical tools others than those described hereinabove, for example one or several osteosynthesis wire(s) 21, for example Kirschner wires, or still one or several percutaneous burr(s) allowing realizing bone cuts.

As illustrated in FIG. 7, said kit 27 is advantageously constituted by a package 46, made for example of a polypropylene-type transparent plastic, in which are disposed said surgical instrument 1, bistoury blade holder handle 28, rugine 29 and rasp 30, these being preferably cushioned in said package 46 by means of a cushioning foam.

Preferably, said surgical kit 27 is intended for a single use, that is to say that said surgical instrument 1, bistoury blade holder handle 28, rugine 29 and rasp 30 composing it are intended to be destroyed or recycled/valorized at the end of their use for a given patient and surgical operation.

However, without departing from the scope of the invention, said kit 27 may on the contrary be reusable, that is to say that said surgical instrument 1, bistoury blade holder handle 28, rugine 29 and rasp 30 composing it may be supplied again, after a first use, to the surgeon after having undergone a retreatment and an adequate reconditioning.

As such, said surgical instrument 1, bistoury blade holder handle 28, rugine 29 and rasp 30 are advantageously all made of a polymer material, for example a polyacrylamide (PAA) based composite polymer material, the latter being optionally loaded.

Still more preferably, said surgical instrument 1, bistoury blade holder handle 28, rugine 29 and rasp 30 are advantageously conditioned in a sterile manner in the package 46, so as to be immediately usable by the surgeon without a preliminary cleaning and sterilization step.

Finally, the invention may concern, as such, a method for using the surgical instrument 1 according to the invention in order to modify the relative positioning of bone bodies 2, 3, for example in the context of the surgical treatment of a pathology of the foot of a patient as illustrated in FIG. 6.

Advantageously, such a method comprises at least the following successive steps of:
incising the body of the patient, typically over a few millimeters (for example about 2 to 5 mm) in order to enable introducing at least partially the working head 8 of the surgical instrument 1 inside, at the level of the bone bodies 2, 3, for example at the level of a first bonny segment 2 formed by the body of a first metatarsus and a second bonny segment 3 formed by the head of this first metatarsus;
spreading said bone bodies 2, 3 apart from each other using the working head 8, in order to insert the working tip 15 of the working head 8 in the medullary cavity 4 of a first bone body 2;
continuing spreading and displacing said bone bodies 2, 3 relative to each other while bearing on the second bone body 3, preferably by making the concave portion of the upper surface 12 fitted with longitudinal flanges 25 cooperate with the surface of the second bone body 3, and by exerting a lever effort on the gripping handle 5 until obtaining the desired preferred positioning of said bone bodies 2, 3.

Preferably, said method comprises an additional step, subsequent to those described hereinbefore, in which, while maintaining the lever effort exerted on said bone bodies 2, 3 using the instrument 1 in order to preserve the obtained preferred positioning, an osteosynthesis wire 21 is brought to slide in the groove 16 of said instrument, from the proximal opening towards the distal opening of said groove 16, so as to accurately position and fasten said wire 21 in the medullary cavity of said first bone body 3.

Of course, such a method may be implemented, by percutaneous or [symbol] open [symbol] surgery, in the context of the treatment of a pathology affecting bones others than those of the foot, for example bones of the hand, as long as it is necessary to displace bone bodies 2, 3 in order to modify or correct their positioning.

In fine, the surgical instrument 1 of the invention, particularly ergonomic and simple to use, allows the surgeon to modify the relative positioning of bone bodies 2, 3 of a patient, while advantageously enabling the implementation of an osteosynthesis wire 21 in a particularly accurate manner and without having to remove the surgical instrument 1. Made by a simple manufacturing method, the surgical instrument 1 is relatively inexpensive to produce. Furthermore, it is advantageously integrated in the surgical kit 27 of the invention, so as to offer to the surgeon a complete, ergonomic, safe and inexpensive set of tools intended for the implementation of an operative technique, for example percutaneous, for treating a bone pathology, in particular of the foot.

1. A surgical instrument (1) intended to be maneuvered like a lever in order to allow modifying the relative positioning of bone bodies (2, 3) of a patient, for example of his foot, said surgical instrument (1) including:
a gripping handle (5) extending along a first longitudinal axis (A-A′),
a working head (8), including an elongate body (9) extending along a second longitudinal axis (B-B′) between a first end (10) linked to the gripping handle (5) and a second free end (11), said body (9) presenting an upper surface (12) and a lower surface (13) linking said first (10) and second (11) ends,
said surgical instrument (1) being characterized in that the working head (8) is provided with an axial groove (16) formed in said upper surface (12).

2. The surgical instrument (1) according to claim 1, characterized in that said axial groove (16) extends between a proximal opening (17) and a distal opening (18), said proximal (17) and distal (18) openings opening from the upper surface (12) of the body (9) of said working head (8).

3. The surgical instrument (1) according to claim 1, characterized in that said groove (16) is formed by:
a groove bottom (19) extending, from the proximal opening (17), along the longitudinal axis (B-B′) over at least a portion of the length of the working head (8),
two groove walls (20) rising from the bottom (19) up to the upper surface (12).

4. The surgical instrument (1) according to claim 3 characterized in that said walls (20) are configured so as to confer to said groove (16) a substantially conical profile.

5. The surgical instrument (1) according to claim 1, characterized in that the body (9) of the working head (8) extends:
laterally between two lateral edges (14), and
transversely between said upper (12) and lower (13) surfaces,
said lateral edges (14) and said upper (12) and lower (13) surfaces being respectively substantially parallel over at least a portion of the length of the working head (8), and then convergent towards the free end (11) of the latter so as to form at this free end (11) a working tip (15).

6. The surgical instrument (1) according to claim 1, characterized in that said working head (8) is recurved.

7. The surgical instrument (1) according claim 6, characterized in that the body (9) of the working head (8) extends substantially along a proximal extension plane (Pp) secant to the first longitudinal axis (A-A′), the body (9) of the working head (8) and the gripping handle (5) being linked by a first curvature area (23).

8. The surgical instrument (1) according to claim 6, characterized in that the body (9) of the working head (8) extends substantially along a proximal extension plane (Pp), except its working tip (15) which in turn extends substantially according to a distal extension plane (Pd) secant to the proximal extension plane (Pp), said working tip (15) and said working head (8) being linked by a second curvature area (24).

9. The surgical instrument (1) according to claim 7, characterized in that said proximal opening (17) and distal opening (18) of the groove (16) opening from the upper surface (12) of the body (9) of said working head (8) are positioned respectively at the level of said first (23) and second (24) curvature areas.

10. The surgical instrument (1) according to claim 1, characterized in that the working head (8) is fitted with two longitudinal flanges (25) protruding from the elongate body (9) of said working head (8), so as to prolong said upper (12) and lower (13) surfaces, said longitudinal flanges (25) being disposed symmetrically with respect to an extension plane (Pg) of the groove (16) and radially with respect to the longitudinal axis (B-B′).

11. The surgical instrument (1) according to claim 10, characterized in that said longitudinal flanges (25) are positioned proximate to said first end (10) of the working head (8) and extending longitudinally substantially over half the length of said working head (8).

12. The surgical instrument (1) according to claim 10, characterized in that said longitudinal flanges (25) are incurved so as to locally confer to said upper surface (12) a substantially concave shape and to said lower surface (13) a substantially convex shape.

13. The surgical instrument (1) according to claim 1, characterized in that the first (A-A′) and second (B-B′) axes are coincident.

14. The surgical instrument (1) according to claim 1, characterized in that it is intended for a single use.

15. The surgical instrument (1) according to claim 1, characterized in that said surgical instrument (1) is made of a polymer material, for example a polyacrylamide (PAA) based composite polymer material, the latter being optionally loaded.

16. The surgical instrument (1) according to claim 1, characterized in that said gripping handle (5) and said working head (8) are integral with each other.

17. A method for manufacturing a surgical instrument (1) according to claim 16, characterized in that it comprises one single molding step during which said surgical instrument (1) is integrally made in one single piece.

18. A surgical kit (27) intended for the implementation of an operative technique, preferably percutaneous, in particular in the context of the treatment of a pathology of the foot of a patient, comprising a surgical instrument (1) according claim 1, and being characterized in that it also comprises at least one bistoury blade holder handle (28), a rugine (29) and a rasp (30).

19. The surgical kit (27) according to claim 18, characterized in that it is intended for a single use.

20. The surgical kit (27) according to claim 18, characterized in that said surgical instrument (1), bistoury blade holder handle (28), rugine (29) and rasp (30) are all made of a polymer material, for example a polyacrylamide (PAA) based composite polymer material, the latter being optionally loaded.
